(19) United States
(12) Patent Application Publication
(10) Pub. No.: US 2024/0050047 A1
(43) Pub. Date: Feb. 15, 2024

(54) REAL-TIME MARKER-LESS DYNAMIC TUMOR TRACKING

(71) Applicant: THE BOARD OF REGENTS OF THE UNIVERSITY OF TEXAS SYSTEM, Austin, TX (US)

(72) Inventors: Yiping SHAO, Missouri City, TX (US); Yuncheng ZHONG, Flower Mound, TX (US); Xinyi CHENG, Lewisville, TX (US)

(73) Assignee: THE BOARD OF REGENTS OF THE UNIVERSITY OF TEXAS SYSTEM, Austin, TX (US)

(21) Appl. No.: 18/269,859

(22) PCT Filed: Dec. 31, 2020
(86) PCT No.: PCT/US2021/065773

§ 371 Date: Jun. 27, 2023

Related U.S. Application Data
(60) Provisional application No. 63/132,758, filed on Dec. 31, 2020.

(51) Int. Cl. A61B 6/03; A61B 6/12; A61B 6/00

(52) U.S. Cl. CPC A61B 6/037; A61B 6/12; A61B 6/542; A61B 6/4208

(57) ABSTRACT

Disclosed embodiments include devices, systems, and methods for real time marker less tumor tracking (RMTT). In various embodiments, the RMTT embodiments use positron emission tomography (PET) and time of flight data to determine a 3D position of the tumor. The 3D position of the tumor may be continuously determined multiple times per section during a radiation therapy session to track the position of a moving tumor in real time. The real time tumor tracking data may be used to position a radiation source to deliver radiation directly to the tumor at all times during radiation therapy to improve the effectiveness of radiation treatments.

### PRIORITY CLAIM

This application claims priority pursuant to 35 USC § 119(e) to U.S. provisional patent application No. 63/132,758 filed Dec. 31, 2020. The aforementioned application is hereby incorporated by reference as though fully set forth.

### FIELD OF DISCLOSURE

The present disclosure relates generally to radiation therapies for medical conditions, and, more specifically, to tracking the position of moving tumors in real time to provide accurate radiation target positions for delivering precise external radiation beams to the moving tumors.

### BACKGROUND

Radiation therapy (RT) is commonly used to treat cancer and other deadly diseases. Radiation doses administered during RT treatments can have serious side effects along with their therapeutic properties. To minimize the side effects, the amount of radiation and the dose's distribution inside the patient's body must be carefully planned and managed during a course of RT. To achieve the highest therapeutic ratio, RT treatments aim to irradiate the targeted tumor volume with the prescribed high dose while maintaining the lowest possible dose to the surrounding normal tissues.

Current state of the art RT technology can reliably plan and accurately deliver radiation beams to stationary tumor volumes with accurate position within the specified treatment field margins. However, tumors that move over the course of a treatment fraction, for example, tumor volumes within lung tissue that move constantly due to a respiratory activity of the patient, are much more difficult to accurately irradiate. For moving tumors, larger treatment field margins are usually added to the clinical target tumor volume in order to completely cover the tumor volume across its full range of motion. The inflated treatment margins reduce the effectiveness of RT by increasing the exposure of normal tissues to toxic radiation and reducing the benefit of dose escalation. Therefore, it would be desirable to have a system that reliably tracks the position of a moving tumor in real time that can dynamically adjust or modify the target position of a radiation beam during a radiation therapy session (i.e., an RT session) based on the current tumor position.

Current methods of accounting for the movement of moving tumors are severely limited. One strategy to minimize the impact of respiratory motion is requiring patients to hold their breath during treatment. This method, however, is prone to human error as some patients may take involuntary breaths during treatment and or some residual tumor motion may occur even when the patient is not breathing. Additionally, some patients cannot hold their breath long enough to provide sufficient time for RT.

More complicated marker-based tracking methods have also been developed. Marker-based methods use fiducial markers to detect the respiratory pattern and activate the radiation source only during a specific respiratory phase to account for tumor motion. Respiration, however, is difficult to continuously monitor accurately and most marker-based methods either assume the respiratory pattern stays constant or predict changes in the respiratory pattern based on models. Neither solution can accurately track the position of a moving tumor on a reliable basis, therefore, marker-based methods still result in reduced RT effectiveness. Additionally, implanted fiducial markers can migrate during treatment and marker-gating guided RT prolongs treatment time. Other marker-based imaging methods that measure the movement of skin and body contours to correlate with the internal tumor motion have been developed, but these methods are also inaccurate and unreliable.

### SUMMARY

In one aspect, disclosed herein are methods for real time marker less tumor tracking, the methods comprising determining a volume of interest that includes an area covered by a full range of motion of a tumor that moves inside a patient, wherein the volume of interest is divided into a plurality of regions. Embodiments may also include receiving positron emission tomography (PET) detection data from one or more PET detectors. Embodiments may also include acquiring, from the PET detection data, a plurality of coincidence events provided by a radiotracer substance absorbed by the tumor, wherein each coincidence event included in the plurality of coincidence events produces multiple coincident gamma ray interactions that are detected by the one or more PET detectors. Embodiments may also include determining a line of response for the multiple coincident gamma ray interactions generated by each coincidence event and measuring a time of flight for each gamma emission. Embodiments may also include for each coincidence event, determining an annihilation position for a particular coincidence event along the line of response by measuring a difference between the time of flight for the multiple coincident gamma ray interactions produced during a particular coincidence event. Embodiments may also include determining a distribution of annihilation positions over the volume of interest by counting a number of annihilation positions within each region in the plurality of regions. Embodiments may also include determining a current position of the tumor based on the distribution of annihilation positions.

In some embodiments the plurality of coincidence events are acquired during a measurement time frame having a duration of at least 0.01 seconds. In some embodiments, the current position of the tumor is continuously determined during a radiation therapy session to track a position of the tumor as the tumor moves within the patient.

Embodiments may also include applying a count threshold to the number of annihilation positions within each region of the volume of interest to remove background noise that may reduce an accuracy of the current position of the tumor. Embodiments may also include transmitting the current position of the tumor to a radiation source and controlling a position of the radiation source based on the current position of the tumor to deliver radiation directly at the current position of the tumor.

In some embodiments the line of response corresponds to a straight line drawn between two gamma rays detected by the one or more PET detectors during a particular coincidence event. In some embodiments, the annihilation positions are three dimensional and include at least one coordinate indicating a location in a vertical direction, horizontal direction, and depth direction relative to a geometric center of the patient. In some embodiments, the tumor is within an organ that moves during a respiratory activity of the patient. In some embodiments, the distribution of annihilation positions and the current position of the tumor are generated from the PET detection data in less time and using fewer computational resources than are required to construct and image representation of the PET detection data.

Embodiments may also include using the current position of the tumor to improve a quality of an image representation of the tumor generated using stationary PET imaging.

In one aspect, disclosed herein are tumor tracking devices comprising one or more PET detectors arranged in a partial ring structure that is configured to mount to a linear accelerator (LINAC) that contains a radiation source. Embodiments may also include a time of flight sensor coupled to the one or more PET detectors, a memory, and a processor circuit configured to execute one or more lines of instructions stored in memory. In some embodiments the processor circuit is configured to determine a volume of interest that includes an area covered by a full range of motion of a tumor that moves inside a patient, wherein the volume of interest is divided into a plurality of regions. In some embodiments, the processor circuit is further configured to receive positron emission tomography (PET) detection data from the one or more PET detectors; and acquire, from the PET detection data, a plurality of coincidence events provided by a radiotracer substance absorbed by the tumor, wherein each coincidence event included in the plurality of coincidence events produces two coincident gamma ray interactions that are detected by the one or more PET detectors. In some embodiments, the processor circuit is further configured to determine a line of response for the two coincident gamma ray interactions generated by each coincidence event and measure, using the time of flight sensor, a time of flight for each coincident gamma ray interaction. In some embodiments, the processor circuit is further configured to for each coincidence event, determine an annihilation position for a particular coincidence event along the line of response by measuring a difference between the time of flight for the two coincident gamma ray interactions produced during a particular coincidence event and determine a distribution of annihilation positions over the volume of interest by counting a number of annihilation positions within each region in the plurality of regions. In some embodiments, the processor circuit is further configured to determine a current position of the tumor based on the distribution of annihilation positions.

In some embodiments, the plurality of coincidence events are acquired during a measurement time frame having a duration of between 0.01 seconds and 0.2 seconds. In some embodiments, the current position of the tumor is continuously determined during a radiation therapy session to track a position of the tumor as the tumor moves within the patient. In some embodiments, the processor circuit is further configured to apply a count threshold to the number of annihilation positions within each region of the volume of interest to remove background noise that may reduce an accuracy of the current position of the tumor. In some embodiments, the processor circuit is further configured to transmit the current position of the tumor to a radiation source and control a position of the radiation source based on the current position of the tumor to deliver radiation directly to the tumor.

In some embodiments, the line of response corresponds to a straight line drawing between two gamma rays detected by the one or more PET detectors. In some embodiments, the annihilation positions are three dimensional and include at least one coordinate indicating a location a vertical direction, horizontal direction, and depth direction relative to a geometric center of the patient. In some embodiments, the tumor is within an organ that moves during a respiratory activity of the patient. In some embodiments, the distribution of annihilation positions and the current position of the tumor are generated from the PET detection data in a timer period of less than a second and using fewer computational resources than are required to construct and image representation of the PET detection data. In some embodiments, the processor circuit is further configured to use the current position of the tumor to improve a quality of an image representation of the tumor generated using stationary PET imaging.

Disclosed herein are methods of tracking a moving tumor (or tumors) in real time (i.e. with multiple measurement time frames per second) without any fiducial marker (i.e. marker-less) based on the detected Time-Of-Flight (TOF) information from in-vivo, real-time data acquired by a positron emission tomography (PET) is disclosed herein. Positron emission radiotracer may be injected to and distributed inside the patient as a standard PET imaging procedure before the radiation therapy session. During the radiation therapy session and within each timeframe, PET will acquire coincidence events data, calculate TOF from each coincidence event, estimate the positron-electron annihilation position alone its line of response (LOR), and form a distribution of such positions calculated from all LORs that correspond to the radiotracer uptake distribution. From this estimated distribution of positron-electron annihilation positions, the tumor volume may be identified from as having the higher density of positions relative to the neighboring area. The tumor volume may be identified by applying an appropriate volume of interest (VOI) and threshold of density, calculating the weighted mean of the positions within the VOI and outputting the weighted mean of the positions as the estimated tumor position. All above data processing will be processed online within one timeframe. No conventional image reconstruction is required or applied. These data acquisitions and data processes will be continuously processed over consecutive different timeframes during the radiation therapy session to provide calculated tumor positions over its course of motion for the real time marker-less tracking of the moving tumor.

In some embodiments, systems of real time, marker-less tumor tracking are disclosed. The system includes a modified PET detector including dual detector panels or other detector configurations and can be integrated onboard with an existing typical conventional LINAC radiation therapy system that has a radiation source, such as X-ray source, rotating around a patient to deliver planned therapeutic radiations to the targeted tumor volume. The system may perform one or more operations, which include detecting the gamma ray interactions from the radiotracer and selecting the coincidence events. The one or more operations may further include online calculating TOF from each coincidence event, estimating the positron-electron annihilation position of each coincidence event based on its TOF and LOR information, and creating a distribution of the positron-electron annihilation positions from processing all acquired coincidence events within one timeframe. The one and more operations may further include identifying the tumor volume from the distribution by applying a VOI and threshold of density and calculating the tumor position as the weighted mean over the counts within the VOI. The one and more operations may further include repeating the data acquisitions and tumor position calculations over different timeframes and providing the tumor positions over different timeframes to provide real time marker-less tumor tracking that will lead to a dynamic, adaptive modification of radiation targeting to the moving tumor for improving the radiation therapy accuracy.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1 depicts an exemplary linear accelerator (LINAC) system with a real-time marker-less tumor tracking device for real-time tumor tracking, according to embodiments of the disclosure.

FIG. 2 illustrates an exemplary system for real-time marker-less tumor tracking, according to embodiments of the disclosure.

FIG. 3 illustrates an exemplary tumor tracking device, according to embodiments of the disclosure.

FIG. 4 illustrates an exemplary line of response, according to embodiments of the disclosure.

FIG. 5 illustrates exemplary distribution of annihilation positions obtained using the real-time marker-less tumor tracking system, according to embodiments of the disclosure.

FIG. 6 illustrates the detected tumor positions relative to the physical tumor positions, according to embodiments of the disclosure.

FIG. 7 is a flow chart illustrating an exemplary process for tracking a tumor using the real-time marker-less tumor tracking system, according to embodiments of the disclosure.

FIG. 8 is a flow chart illustrating an exemplary process for administering RT using the real-time marker-less tumor tracking system, according to embodiments of the disclosure.

FIG. 9 is a flow chart illustrating an exemplary process for administering RT based a location of a line of response, according to embodiments of the disclosure

FIG. 10 is a block diagram of an illustrative computer device that may be used to implement the system of FIG. 2, according to embodiments of the disclosure.

### DETAILED DESCRIPTION

Disclosed herein are systems and methods for real-time marker-less tumor tracking (RMTT). The RMTT techniques described herein detect the position of a tumor in real-time based on time of flight (TOF) information collected using positron emission tomography (PET) detectors. The TOF information may be used to determine an annihilation position (i.e., a location where positron-electron annihilation occurs and the annihilation generates a pair of coincident 511 keV gamma rays) along a line of response that corresponds to the position of the radioisotope uptaken by the tumor or other normal tissues. The PET detectors may be mounted to any existing LINAC system so that the dual PET detectors rotate simultaneously with the LINAC during RT. Many annihilation positions are determined by the PET detectors within a measurement time frame. A distinguishable distribution of annihilation positions that has higher count density within the distribution and lower count density outside the distribution may be identified as the annihilation positions associated with the tumor. Once the RMTT system determines the tumor position based on the annihilation positions, the RMTT system will determine the tumor position changes due to tumor motion. The RMTT system can then determine how to adjust the radiation target position based on the change in tumor position. The LINAC can then align with the updated radiation target position and deliver radiation beams to the updated target position. The RMTT may determine the position of the tumor from the distribution of annihilation positions continuously during the RT session at near real time intervals (e.g., between 0.01 s-0.2 s time frames) to track the tumor in real time and provide the updated target positions for the LINAC.

The disclosed RMTT techniques improve upon existing technology be providing 3D tumor position data based on TOF information. The TOF information may be collected directly from the acquired PET projection data. Other tracking techniques including X-ray planar imaging and MR-LINAC are limited to planar positional data, therefore cannot accurately perform 3D tumor tracking. The inability of these systems to generate 3D positional data limits the tracking accuracy particularly for irregular shaped tumors. The disclosed RMTT techniques generate 3D tumor tracking data from raw TOF and PET detection information and therefore do not require image reconstruction. The ability to use raw TOF and PET detection information dramatically increases the speed at which tumor position can be calculated. Other tracking techniques including on-board CT, CBCT imaging, and PET imaging move too slow (e.g., a slow LINAC gantry motion) or require too much time for data acquisition and image reconstruction to allow for real-time tumor tracking. TOF and PET data acquisition is also relatively safe and convenient for the patient compared to methods that rely on high intensity radiation (e.g., X-ray planar imaging) or invasive procedures (e.g., PET using an implanted tumor marker).

Reference will now be made in detail to embodiments, examples of which are illustrated in the accompanying drawings. In the following detailed description, numerous specific details are set forth in order to provide a sufficient understanding of the subject matter presented herein. However, it will be apparent to one of ordinary skill in the art that the subject matter may be practiced without these specific details. Moreover, the particular embodiments described herein are provided by way of example and should not be used to limit the scope of the invention to these particular embodiments. In other instances, well-known components have not been described in detail so as not to unnecessarily obscure aspects of the embodiments of the invention.

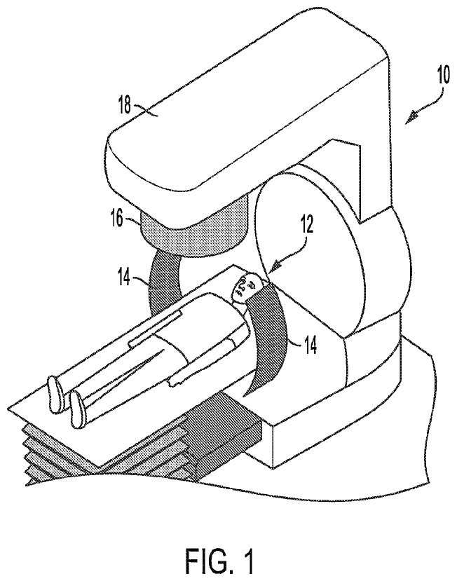

FIG. 1 illustrates an exemplary LINAC system 10 for RMTT. The LINAC system 10 may include a partial ring structure 12 having dual PET detectors (or detector panels) 14. The partial ring structure 12 may be configured as an add-on component that may be mounted to an existing LINAC to provide an integrated PET-LINAC as shown in the LINAC system 10. The LINAC system 10 having the integrated PET-LINAC provides a compact system with simplified real-time data acquisition and processing. The compatibility of the partial ring structure 12 with existing LINACs enables the RMTT method described herein to be implemented without expensive and lengthy development of new technology and or LINAC equipment.

The dual PET detectors 14 may be stationary relative to LINAC system 10 and may remain in place during the RT session even as the radiation source 16 moves about the patient to optimize the radiation delivery position and angle. The dual PET detectors 14 may also be configured to rotate simultaneously with the radiation source 16 around the patient. The LINAC system 10 may include a rotating gantry 18 to rotate and otherwise move the radiation source 16 and our dual PET detectors 14 around the patient. Regardless of whether the dual PET detectors 14 are configured to be stationary or mobile, the dual PET detectors 14 acquire sufficient coincidence event detection data and TOF information to enable RMTT. The flexibility of the dual PET detectors 14 increases the interoperability of the RMTT system described herein with existing technology an ensures the partial ring structure 12 can function with any existing LINAC system.

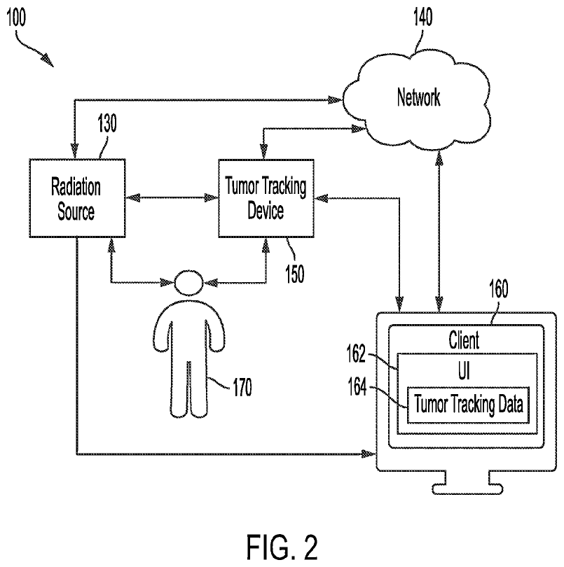

FIG. 2 illustrates a system for RMTT 100. The system 100 may include a radiation source 130 (e.g., a radiation x-ray beam within LINAC) that delivers radiation to a patient 170 during an RT session. The radiation source 130 may be coupled to a tumor tracking device 150 (e.g., dual PET detectors) via a wired and or wireless connection (e.g., connection over a network 140). The radiation source 130 and tumor tracking device 150 may be integrated into one device as shown in the LINAC system of FIG. 1. The radiation source 130 and the tumor tracking device 150 may also be configured as separate, stand-alone devices.

The tumor tracking device 150 may detect tumor tracking data 164, for example, TOF information and coincidence event lines of response and other detection data measured by dual PET detectors shown in FIG. 1. The tumor tracking device may determine the position of the tumor from the tumor tracking data 164 and communicate the position of the tumor to the radiation source 130. To maximize the effectiveness of RT, the radiation source 130 may move relative to the patient 170 based on the position of the tumor to deliver radiation to a portion of the patient 170 that contains the tumor. During an RT session, the tumor tracking device 150 may constantly measure tumor tracking data 164 and or determine the position of the tumor. For example, the tumor tracking device 150 may measure tumor tacking data 164 at a sample rate of hundreds or thousands of time per second. Accordingly, the measurement time frame may be any time period of less than one second or longer. During each measurement time frame (e.g., 0.01 s to 0.2 s), the tumor tracking device 150 may determine the position of the tumor and or whether the determined tumor position satisfies one or more conditions (e.g., conditions for accuracy, reliability, precision, and the like). If the one or more conditions are satisfied, the tumor tracking device 150 may communicate the tumor position to the radiation source 130 to position the radiation beam relative to the latest tumor position within the patient. Once in position, the radiation source 130 may actuate to deliver radiation to the portion of the patient 170 that contains the tumor. This process of measuring tumor tracking data 164, determining the tumor position, verifying the determined tumor position for one or more conditions, communicating the tumor position to the radiation source, moving the radiation source to the new radiation target position, and actuating the radiation beams may repeat constantly several times per second during the RT session to provide dynamic, real time (e.g., 0.01 s to 0.2 s) tumor tracking and positioning of the radiation target to match the position of the tumor as the tumor moves inside the patient 170.

Tumor tracking data 164 measured by the tumor tracking device 150 may also be provided to a client device 160. Client device(s) 160 may be any device configured to present user interfaces (UIs) 162 and receive user inputs thereto. The client device 160 may be, for example, a desktop computer, laptop computer, smartphone, tablet, mobile device, monitor, or any other piece of electronics equipment configured to manipulate and or display data. The client device 160 may provide the UI 162 to display the tumor tracking data 164 to a user (e.g., a patient, doctor, radiation technician, and or other healthcare provider). The client device 160 may also determine the position of the tumor based on the tumor tracking data 164 and may display the position of the tumor to a user via the UI 162.

The radiation source 130, tumor tracking device 150, and client device 160 are each depicted as single devices for ease of illustration, but those of ordinary skill in the art will appreciate that radiation source 130, tumor tracking device 150, and client device 160 may be embodied in different forms for different implementations. For example, any or each of radiation source 130, tumor tracking device 150, and client device 160 may include a plurality of components, devices, and or other pieces of equipment. Alternatively, the operations performed by any or each of the radiation source 130, tumor tracking device 150, and client device 160 may be performed on fewer devices. In another example, a plurality of client devices 160 may communicate with the radiation source 130 and or tumor tracking device 150. A single user may have multiple client devices 160, and or there may be multiple users each having their own client device(s) 160.

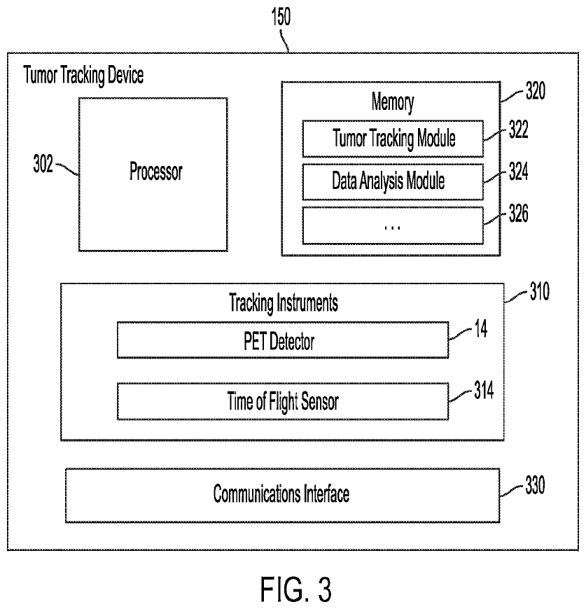

FIG. 3 depicts a block diagram illustrating more details of the tumor tracking device 150 shown in FIG. 2. The tumor tracking device 150 may include a processor 302, a memory 320, one or more tracking instruments 310, and a communications interface 330. The memory 320 may store one or more modules including one or more lines of instructions executable by the processor 302 to perform any suitable operation or combination of operations to provide the functionalities of the tumor tracking device 150.

The processor 302 can include one or more cores and can accommodate one or more threads to run various applications and modules. Software can run on the processor 302 capable of executing computer instructions or computer code. The processor 302 can also be implemented in hardware using an application specific integrated circuit (ASIC), programmable logic array (PLA), field programmable gate array (FPGA), or any other integrated circuit. The processor 302 may be configured to perform general purpose computations and communications of the tumor tracking device 150. For example, the processor 302 may receive and process data (e.g., tumor tracking data) measured by the tracking instruments 310 and other sensors within the tumor tracking device 150.

Memory 320 can be a non-transitory computer readable medium, flash memory, a magnetic disk drive, an optical drive, a programmable read-only memory (PROM), a read-only memory (ROM), or any other memory or combination of memories. Memory 320 can include one or more modules, for example, a tumor tracking module 322, a data analysis module 324, and a plurality of other modules 326. The processor 302 can be configured to run one or more modules stored in memory 320 that are configured to cause the processor 302 to perform various steps that are discussed throughout the present disclosure.

The tumor tracking device 150 may include one or more tracking instruments 310 configured to measure tumor tracking data. The tracking instruments 310 may include one or more dual PET detectors 14 configured to detect gamma rays emitted by radiotracers that accumulate around and or are absorbed by tumor cells inside the patient. The radiotracers may emit a positron and consequently produce two gamma rays after an annihilation of a positron and an electron (i.e., an annihilation event). The annihilation event may be detected by the PET detectors 14 as a coincidence event. A detected coincidence event may form a line of response (LOR) that is a straight line drawn between the two detected gamma rays. The tracking instruments 310 may also include a TOF sensor or signal processor 314 that determines a TOF for the coincident gamma rays detected by the PET detector 14 to determine an annihilation position along a line of response. The processor 302 may receive TOF measurements from the time of flight sensor or processor 314 and use the tumor tacking module 322 to determine the location of the annihilation event (i.e., an annihilation position) within a volume of interest for the tumor based on the TOF measurements and the line of responses detected from PET detection data.

In various embodiments, the tumor tracking device 150 may communicate with external devices (e.g., a radiation source, client device, and the like) via a communications interface 330. The communications interface 330 may be a wireless interface. The wireless interface may be a standard Bluetooth® (IEEE 802.15) interface, such as Bluetooth® v4.0, also known as “Bluetooth low energy.” In various embodiments, the interface may operate according to a network protocol such as, and Internet Protocol (IPv4, IPv6, and the like), a cellular network protocol (e.g., Long Term Evolution (LTE™)), or a Wi-Fi (IEEE 802.11) protocol. In various embodiments, the communications interface 330 may include wired interfaces, such as an Ethernet cable, headphone jack, USB connector, or a bus connector.

The components described above are examples, and embodiments of tumor tracking device 150 may include other components not shown. Additionally, embodiments of the tumor tracking device 150 may include fewer components that those pictured in FIG. 3. In some embodiments, all components within the tumor tracking device 150 can be electrically and/or mechanically coupled together. In some embodiments, the processor 302 can coordinate the communication among each component.

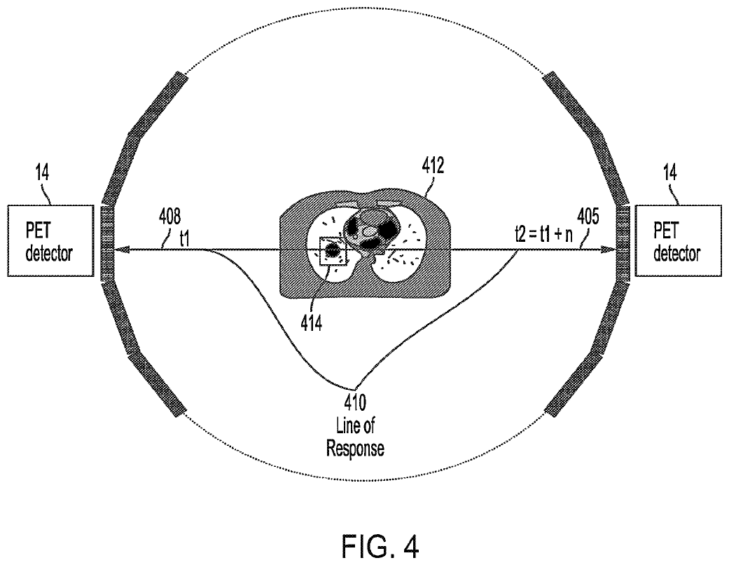

FIG. 4 illustrates and exemplary line of response 410 and TOF data used to determine the position of a tumor inside the body of a patient. The line of response 410 may be determined based on PET detection techniques. For example, the line of response 410 may be determined based on the acquisition of a coincidence interaction event of two 511 KeV gamma rays produced from the annihilation of a positron-electron pair (e.g., an annihilation event or coincidence event) that occurred at or near a radiotracer bound to and or absorbed by a tumor (414). The line of response 410 is a straight line drawing between the detected two coincidence gamma ray interactions by the PET detectors 14. The gamma rays may be detected by the dual PET detectors 14 shown in FIG. 1. To generate annihilation events located at the tumor 414, a radiotracer (e.g., ¹⁸F-FDG or any other radiotracer substance) may be injected into a patient. The radiotracer may be the combination of a radioactive isotope of an element (e.g., fluorine-18) and a molecule that readily binds to cells having particular characteristics, for example, ¹⁸F combined with fluorodeoxyglucose. ¹⁸F concentrates at or around tumor cells are higher because tumor cells have a relatively high metabolic rate relative to normal issues therefore have a greater demand for and affinity to glucose. The distribution of ¹⁸F-FDG and other radiotracers used during PET scans typically settles in high concentrations at the tumor 414 location.

The dual PET detectors 14 used to acquire gamma rays emitted during an annihilation event may be configured in a rotating two half-ring arrangement. The distance between the dual PET detectors may be, for example, 95 cm. Each detector panel may include five detector modules that include a 50×84 array of 3×3×20 mm³ lutetilm-yttrnum oxyorthosilicate (LYSO) scintillators. The detector modules may be, for example, 15 cm by 25.2 cm (tangential×axial). The detectors may determine a line of response by drawing a straight line between the two coincident gamma ray interactions detected by the dual detectors.

The annihilation positions of a coincidence event which is along the line of response can be determined based on the TOF information from the two detected coincident gamma rays generated by the annihilation. To determine the difference (n) between the detected time of a first gamma ray 408 (t1) and a second gamma ray 406 (t2), the time for the first gamma emission 408 may be subtracted from the time of for the second gamma emission 406 (or vice versa). The difference between the two detected gamma ray interaction times, which is also the difference between the times of flight for the two detected gamma rays traveling from the annihilation position to their respected detectors, is shown by equation 1 below.

n=T₂−T₁ Equation 1:
Where T₁ is the detected time for the first gamma ray; T₂ is the detected time for the second gamma ray; and n is the difference between T1 and T2 that is also the difference in time of flight between the two gamma rays.

This difference in time of flight is used to determine the annihilation position along the line of response. For example, if the difference in time of flight (i.e. n) between the first detected gamma ray 408 and the second detected gamma ray 405 along the line of response 410 is 100 picoseconds, the annihilation position is 1.5 cm away from the center of line of response 410 toward the detector that detected the gamma ray first in time. The distance from the center of line of response 410 to the annihilation position may be derived from the difference in time of flight based on equation 2 shown below.

Δx=(Δt)×C/2 Equation 2:
Where Δx=the distance between the center of line of response and the annihilation position, Δt=n, and C=speed of light.
   All above are known knowledge. The essence of the invention is that one can use Equation 2 to directly calculate annihilation positions without applying any tomographic image reconstruction. Based on these calculated annihilation positions, one can then identify a tumor, calculate its position, and track its motion in real-time.

The accuracy of annihilation positions based on TOF and line of response depends mainly on the coincidence time resolution of the dual PET detectors 14. As shown above, even a 100 picosecond order of magnitude error in detection can misplace the annihilation position by around 1.5 centimeters. Therefore, even for data acquired from a single point radioisotope, the distribution of annihilation positions calculated from time of flight data may be blurred from their original physical annihilation positions due to limited PET coincidence time resolution. However, the center of the calculated annihilation positions related to the tumor will not change significantly if the spatial blurs are about the same in terms of the scale and variation along different lines of response. In other words, as long as the time of flight resolution is consistent for all detections during an acquisition, each calculated annihilation position will be impacted by the same spatial blurring effect in terms of the scale and variation so that the center of the calculated annihilation positions will be about the same as the physical position of the tumor.

Therefore, the three dimensional (3D) mean position of the tumor 414 may be estimated based on the corresponding calculated annihilation positions within the tumor boundaries (i.e., a volume of interest that covers an area that includes the full range of motion of a tumor) within each measurement time frame. The 3D mean position may include one or more coordinates that describe the location of the tumor in three coordinate directions. For example, the 3D mean position of the tumor may include at least one coordinate indicating a location of the tumor in a vertical direction (i.e., along a y axis and or up and down relative to the geometric center of the patient), at least one coordinate indicating a location of the tumor in a horizontal direction (i.e., along an x axis and or left and right relative to the geometric center of the patient), and at least one coordinate indicating a location of the tumor in a depth direction (i.e., along a z axis and or in towards the geometric center of the patient and out from the geometric center of the patient).

A predetermined volume of interest (VOI) may be used to define the tumor boundaries for selecting the data related to the tumor and improving the accuracy in calculating the tumor position. The VOI may be used to reject background counts (i.e., gamma detections) so that only the calculated annihilation positions within the VOI may be selected for further processing. The VOI boundaries may be set to cover and follow the entire tumor volume over the course of the full range of tumor motion. To facilitate more precise tumor tracking, the VOI may be divided into voxels and the counts of annihilation positions (i.e., the number of annihilation positions) inside each voxel may be accumulated during each measurement time frame (e.g., 0.01 s to 0.2 s). At the end of each measurement time frame, the counts of annihilation positions may be counted. To improve the accuracy in calculating tumor position, a count threshold may be applied to the counts within the VOI voxels to remove background noise and select only calculated annihilation positions that are detected with primary importance contributions during the measurement time frame for tumor location. In one embodiment, the count per voxel threshold may be 50% of the maximum count per voxel. For example, the voxel threshold value may be set to one thousand count per voxel (i.e., the highest count per voxel among all voxels within the VOI with a particular measurement time frame). In other words, only voxels with their counts equal or above the top of 50 percent will be included in the calculation to determine the mean position of the tumor. The mean position of the tumor may be calculated as the count weighted centroid of the voxels within the VOI using equation 3 below.

$$\overline{x} = \frac{( \sum {x}_{i} \times {C}_{i} )}{\sum {C}_{i}} \qquad Equation 3$$

Where x̅ is the mean position along the x direction; and xᵢ and Cᵢ are the x coordinate of the voxel i and the count within the voxel i respectively.

The mean tumor position along the y and z directions may be similarly calculated to provide truly 3D tumor position that is not limited to a single planar plane or projection. The fast (i.e., multiple frames per second; a measurement time frame having a duration between 0.01 s and 0.2 s) detection of time of flight and line of response data and determination of annihilation position without image reconstruction enable real-time tracking of the tumor 414.

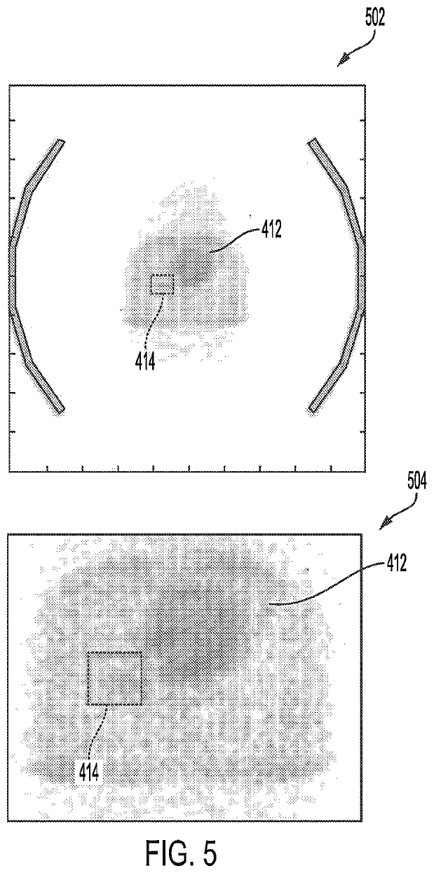

FIG. 5 is a Monte Carlo simulation simulated visual representation of a distribution of annihilation positions determined from PET projection data with TOF. The distribution of annihilation positions shown in FIG. 5 has an 8:1 tumor to background ratio of radiotracer uptake concentrations. The images shown in FIG. 5 illustrate the annihilation positions obtained from PET projection data with TOF resolution equal to 250 picoseconds (ps) and a 350-650 KeV energy window for gamma interactions. The top image 502 illustrates the distribution of annihilation positions detected within one 0.2 s measurement time frame. The bottom image 504 is a zoomed view that focuses on a region of interest that includes the tumor 414. As shown in FIG. 5, the tumor 414 is clearly visible from the real-time calculation based on TOF information from acquired PET projection data and the tumor may be readily distinguished from the background 412 and other normal tissues (i.e., heart and other organs). The tumor 414 shown in this example is a lung tumor that measures 2.8 cm in diameter.

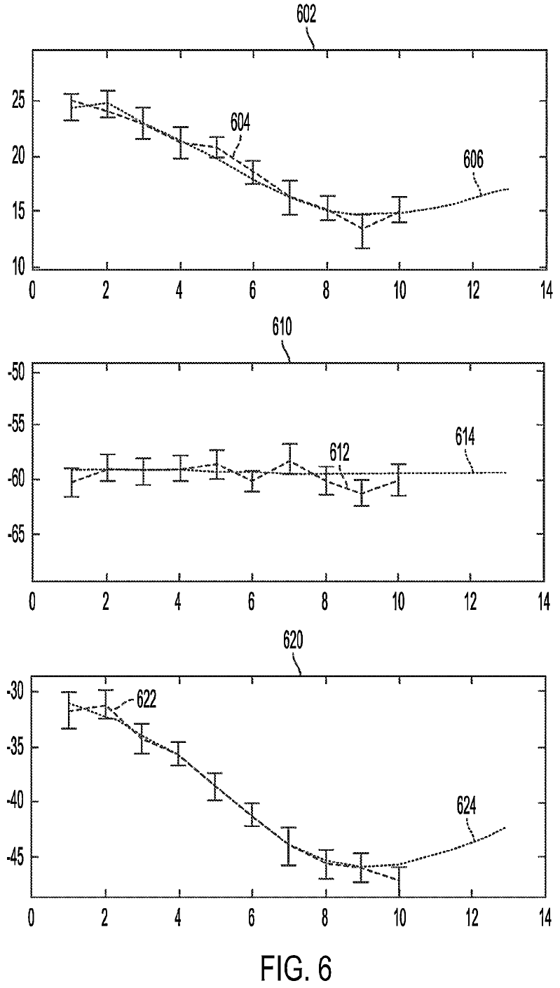

FIG. 6 shows the results of simulation studies that includes three graphs showing the calculated mean tumor positions measured from the TOF of PET projection data relative to the physical tumor motion positions. The top graph 602 illustrates the measured mean tumor position along the x axis (lateral direction; left and right from the geometric center of the patient) 604 relative to the physical tumor position along the x axis 606. The middle graph 610 illustrates the measured mean tumor position along the y axis (vertical direction; up and down relative to the geometric center of the patient) 612 relative to the physical tumor position along the y axis 614. The bottom graph 620 illustrates the measured mean tumor position along the z axis (depth direction; in toward the geometric center of the patient and out away from the geometric center of the patient) 622 relative to the physical tumor position along the z axis 624. The average measurement errors along the x, y, and z directions shown in top graph 602, middle graph 610, and bottom graph 620 are 0.15 mm, −0.40 mm, and 0.39 mm respectively.

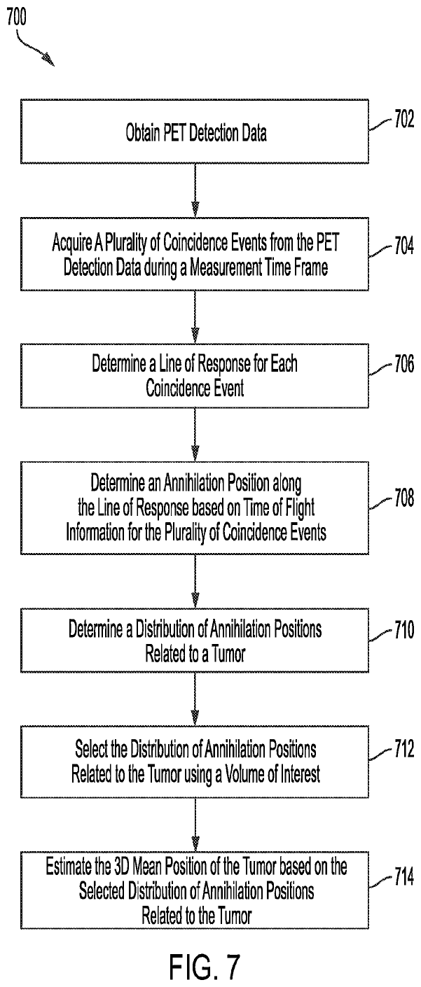

FIG. 7 illustrates an exemplary process for real-time marker-less tumor tracking 700. At 702, PET detection data including gamma rays may be obtained using PET detectors (e.g., the dual pet detectors 14 shown in FIG. 1). At 704, a plurality of coincidence events is acquired from the PET detection data during a measurement time frame. The measurement time frame may be a fraction of one second to enable real-time tumor tracking. For example, the measurement time frame may have a duration between 0.01 seconds (s) and 0.2 seconds (s). The measurement time frame may also be any sub-second time period or longer. At 706, a line of response is determined for each coincidence event. The line of response may be drawn as a straight line between the two coincident gamma ray interactions detected by the dual PET detectors.

At 708, for each coincidence event, an annihilation position is determined along the detected line of response for the event. To determine the annihilation position, time of flight data for the detected coincident gamma rays may be measured by the PET detectors. Based on the time of flight information, the annihilation position of the coincidence event may be located relative to the PET detectors along the line of response. For example, if the time detected by the first PET detector is greater or later than the time detected by the second PET detector, the annihilation position may be determined to be closer to the second PET detector along the line of response. The quantitative calculation is detailed in the above Equations 1 and 2.

The PET detectors may detect hundreds, thousands or even more of coincidence events during a measurement time frame. At 710, a distribution of annihilation positions related to the tumor may be determined for the plurality of coincidence events detected during the measurement time frame. The distribution of annihilation positions may be a representation of the detected distribution of radioisotope uptake concentrations of the tumor and surrounding normal tissues that may be produced in real time without lengthy image reconstruction.

At 712, the distribution of the annihilation positions that are related to the tumor are selected differentiating the higher count density within the distribution relative to the lower count density outside the distribution. A volume of interest may be used to select the volume of this distribution that includes the tumor as well as some unavoidable surrounding normal tissues. To remove the effects from those surrounding normal tissues with relatively low count density, a count threshold may be used to thresh them out, which may improve the accuracy and reliability of the calculated tumor positions. For example, a count threshold may be applied to ensure, for example, only the annihilation positions within the volume of interest with at least 50% of the maximum count per voxel are selected for the further analysis. If the count of annihilation positions does not meet the count threshold, the annihilation positions will not be included in the selected distribution of the annihilation positions related to the tumor used to calculate the 3D mean position of the tumor for the measurement time frame at 714. The 3D mean position of the tumor may represent a current position of the tumor during the measurement time frame. Accordingly, the 3D mean positions of the tumor over the course of RT may be dynamically measured, tracked, and used to dynamically determine the radiation target position for RT. The tumor position data may then be used to position the radiation source to deliver radiation to the tumor.

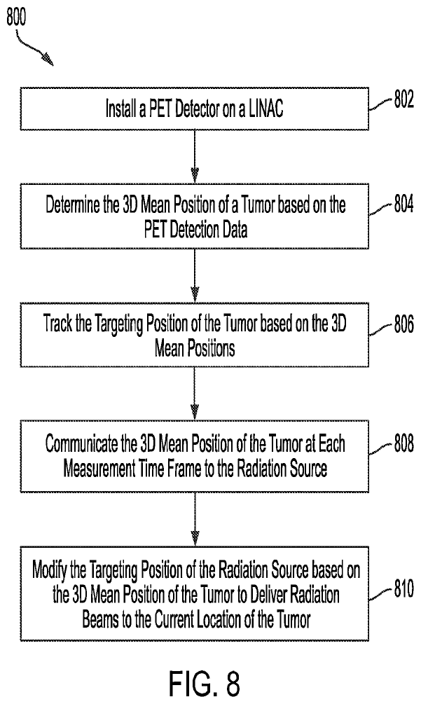

The process for real time marker less tumor tracking may have multiple applications. For example, real time marker less tumor tracking may be used to target the delivery of radiation during RT as described below in FIG. 8. The disclosed real time marker less tumor tracking approach may also be applied to one or more imaging applications (e.g., stationary PET imaging). Acquisition of stationary PET image data does not occur in real time and typically requires a time period of more than one second to several minutes to complete. Therefore, a moving tumor (such as lung and liver tumor) may have several different tumor positions during the PET data acquisition period. These different tumor positions cause the tumor structures to appear blurred in images reconstructed from the PET data and may also produce image motion artifacts. The disclosed process of real time marker less tumor tracking combines PET information with TOF to determine the location of moving tumors in real time. The tumor positions at different acquisition times derived from the PET with TOF data can be used by existing motion compensated image data correction and image reconstruction methods to remove and or minimize the image blurring and or image motion artifacts caused by the tumor motion. Accordingly, the real time marker less tumor tracking techniques described herein can be used to improve the quality of stationary PET images. By increasing the quality of stationary PET images, the real time tumor tracking techniques may help improve the accuracy of image based diagnosis of cancer and other conditions.

FIG. 8 illustrates an exemplary process for administering RT using real time market less tumor tracking 800. At 802, dual PET detectors may be installed on a LINAC. For example, the dual PET detectors 14 shown in FIG. 1 may be installed on a LINAC as part of a partial ring detector system. At 804, the 3D mean position of a tumor may be determined based on PET detection data measured by the PET detectors. For example, the 3D mean position data may be determined based on annihilation positions from the TOF information for coincidence events detected by the PET detectors. The 3D mean position data may include coordinates and or other position data that locates the tumor in three dimensions, for example, along an x, y, and z axis.

At 806, the targeting position of the tumor may be tracked based on the 3D mean positions. The targeting position of the tumor may be tracked in real time by determining the position of the 3D mean position of the tumor every 0.2 s (or any other sub-second or longer time period) from a distribution of annihilation positions. At 808, the tumor tracking device may communicate the 3D mean position of the tumor to a radiation source. For example, the tumor tracking device may send the coordinates corresponding to the 3D mean position of the tumor in a structured data format (e.g., XML file, JSON file, and the like) that may be read by the LINAC. At 810, the target position of the radiation beams may be adaptively modified based on the 3D mean position of the tumor to deliver radiation beams to the current location of the tumor. The tumor tracking device and the LINAC may be in constant communication and or synced so that as the tumor moves the updated 3D mean position data for the tumor is determined by the tumor tracking device, the LINAC receives the updated 3D mean position data and modifies the target position of the radiation beams in real time. To maximize the effectiveness of RT, the real-time tumor tracking data provided by the tumor tracking device may allow the target position of the radiation beams to follow the position of the tumor as the tumor moves in the patient's body.

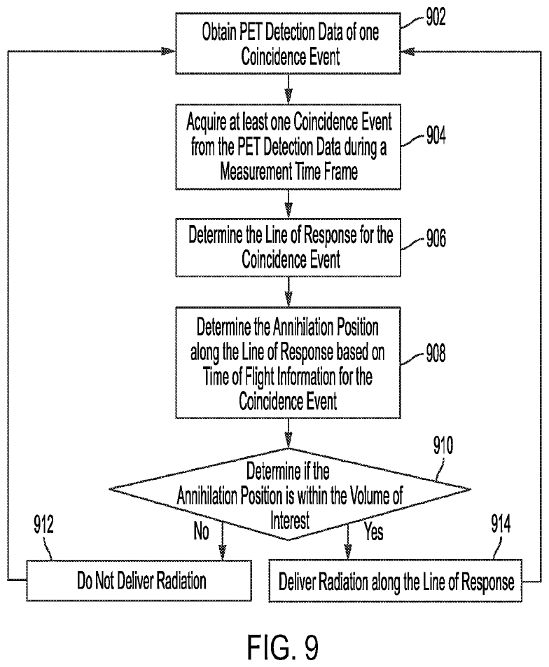

FIG. 9 illustrates an alternate process for administering RT based on the detected line of response. At 902, PET detection data including gamma rays may be obtained using PET detectors (e.g., the dual pet detectors 14 shown in FIG. 1). At 904, at least one coincidence event is acquired from the PET detection data during a measurement time frame as described above in FIG. 7. At 906, a line of response is determined for each coincidence event. For example, the line of response may be drawn as a straight line between the two coincident gamma ray interactions detected by the dual PET detectors.

At 908, for each coincidence event, an annihilation position is determined along the detected line of response for the event as described above in FIG. 7. In various embodiments, the annihilation position determined by time of flight can be used to decide if the detected line-of-response should be selected to guide the radiation or not. At 910, to more precisely target the beam of radiation, the location of the annihilation position may be compared to a volume of interest. The volume of interest may be a pre-determined area that includes the tumor and/or the unavoidable healthy tissues immediately adjacent to the tumor. The volume of interested may be selected based on a computed tomography (CT) scan, magnetic resonance imaging (MRI) scan, or other medical imaging technique and/or determined based on the PET detection data. If the determined annihilation position is within a selected volume of interest, it can be determined that the gamma rays related to the detected line of response were likely originated inside the tumor. Otherwise, if the determined annihilation position is outside the volume of interest, the gamma rays related to the detected line of response were likely originated outside the tumor.

The location of the annihilation position relative to the volume of interest may be used to more intelligently determine if a radiation beam should be delivered along a particular line of response direction. For example, if the annihilation position is within the volume of interest (Yes at 910), a radiation beam is delivered along the line of response direction at 914. If the annihilation position is outside the volume of interest (No at 910), no radiation beam is delivered at 912. Once the decision to deliver radiation or not is made at steps 912-914, the radiation beam targeting process may continue by repeating steps 902-908 to determine an annihilation position for a new measurement time and repeating steps 910-914 to determine whether or not to deliver radiation at the new annihilation position. This process may continue until the tumor may be targeted with sufficient confidence to administer the radiation

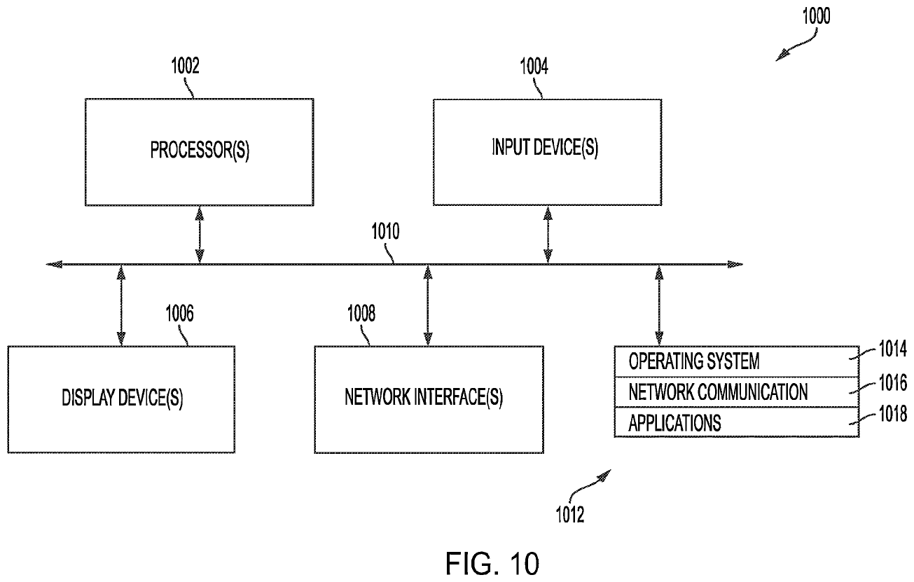

FIG. 10 shows a computing device 1000 according to an embodiment of the present disclosure. For example, computing device 1000 may function as client 160 (which may include a system for analyzing and/or displaying tumor tracking data). The computing device 1000 may be implemented on any electronic device that runs software applications derived from compiled instructions, including without limitation personal computers, servers, smart phones, media players, electronic tablets, game consoles, email devices, etc. In some implementations, the computing device 1000 may include one or more processors 1002, one or more input devices 1004, one or more display devices 1006, one or more network interfaces 1008, and one or more computer-readable mediums 1012. Each of these components may be coupled by bus 1010, and in some embodiments, these components may be distributed among multiple physical locations and coupled by a network.

Display device 1006 may be any known display technology, including but not limited to display devices using Liquid Crystal Display (LCD) or Light Emitting Diode (LED) technology. Processor(s) 1002 may use any known processor technology, including but not limited to graphics processors and multi-core processors. Input device 1004 may be any known input device technology, including but not limited to a keyboard (including a virtual keyboard), mouse, track ball, camera, and touch-sensitive pad or display. Bus 1010 may be any known internal or external bus technology, including but not limited to ISA, EISA, PCI, PCI Express, USB, Serial ATA or FireWire. Computer-readable medium 1012 may be any medium that participates in providing instructions to processor(s) 1002 for execution, including without limitation, non-volatile storage media (e.g., optical disks, magnetic disks, flash drives, etc.), or volatile media (e.g., SDRAM, ROM, etc.).

Computer-readable medium 1012 may include various instructions 1014 for implementing an operating system (e.g., Mac OS®, Windows®, Linux). The operating system may be multi-user, multiprocessing, multitasking, multithreading, real-time, and the like. The operating system may perform basic tasks, including but not limited to: recognizing input from input device 1004; sending output to display device 1006; keeping track of files and directories on computer-readable medium 1012; controlling peripheral devices (e.g., disk drives, printers, etc.) which can be controlled directly or through an I/O controller; and managing traffic on bus 1010. Network communications instructions 1016 may establish and maintain network connections (e.g., software for implementing communication protocols, such as TCP/IP, HTTP, Ethernet, telephony, etc.).

Application(s) 1018 may be an application that uses or implements the processes described herein and/or other processes. For example, a tumor tracking application that determines the position of a tumor within a patient based on tumor tracking data (e.g., PET detection data) collected by a tumor tracking device. conductivity data visualizations, verifies the accuracy and or reliability of tumor position data, transfers the tumor position data to a radiation source to position the radiation source, and the like. The processes may also be implemented in an operating system. For example, application 1018 and/or operating system may present UIs 162 including tumor tracking data 164 which may include lines or response, annihilation positions, counts of annihilation positions, and other measurements collected by the tumor tracking device and or determined from the measurements collected by the tumor tracking device.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, microcontrollers, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

1. A method for real time marker less tumor tracking, the method comprising:
determining a volume of interest that includes an area covered by a full range of motion of a tumor that moves inside a patient, wherein the volume of interest is divided into a plurality of regions;
receiving positron emission tomography (PET) detection data from one or more PET detectors;
acquiring, from the PET detection data, a plurality of coincidence events provided by a radiotracer substance absorbed by the tumor, wherein each coincidence event included in the plurality of coincidence events produces multiple coincident gamma ray interactions that are detected by the one or more PET detectors;
determining a line of response for the multiple coincident gamma ray interactions generated by each coincidence event;
measuring a time of flight for each gamma emission;
for each coincidence event, determining an annihilation position for a particular coincidence event along the line of response by measuring a difference between the time of flight for the multiple coincident gamma ray interactions produced during a particular coincidence event;
determining a distribution of annihilation positions over the volume of interest by counting a number of annihilation positions within each region in the plurality of regions; and
determining a current position of the tumor based on the distribution of annihilation positions.

2. The method of claim 1, wherein the plurality of coincidence events are acquired during a measurement time frame having a duration of at least 0.01 seconds.

3. The method of claim 1, wherein the current position of the tumor is continuously determined during a radiation therapy session to track a position of the tumor as the tumor moves within the patient.

4. The method of claim 1, further comprising applying a count threshold to the number of annihilation positions within each region of the volume of interest to remove background noise that may reduce an accuracy of the current position of the tumor.

5. The method of claim 1, further comprising:
transmitting the current position of the tumor to a radiation source; and
controlling a position of the radiation source based on the current position of the tumor to deliver radiation directly at the current position of the tumor.

6. The method of claim 1, wherein the line of response corresponds to a straight line drawn between two gamma rays detected by the one or more PET detectors during a particular coincidence event.

7. The method of claim 1, wherein the annihilation positions are three dimensional and include at least one coordinate indicating a location in a vertical direction, horizontal direction, and depth direction relative to a geometric center of the patient.

8. The method of claim 1, wherein the tumor is within an organ that moves during a respiratory activity of the patient.

9. The method of claim 1, wherein the distribution of annihilation positions and the current position of the tumor are generated from the PET detection data in less time and using fewer computational resources than are required to construct and image representation of the PET detection data.

10. The method of claim 1, further comprising using the current position of the tumor to improve a quality of an image representation of the tumor generated using stationary PET imaging.

11. A tumor tracking apparatus comprising;
one or more PET detectors arranged in a partial ring structure that is configured to mount to a linear accelerator (LINAC) that contains a radiation source;
a time of flight sensor coupled to the one or more PET detectors;
a memory; and
a processor circuit configured to execute one or more lines of instructions stored in memory, the processor circuit configured to:
determine a volume of interest that includes an area covered by a full range of motion of a tumor that moves inside a patient, wherein the volume of interest is divided into a plurality of regions;
receive positron emission tomography (PET) detection data from the one or more PET detectors;
acquire, from the PET detection data, a plurality of coincidence events provided by a radiotracer substance absorbed by the tumor, wherein each coincidence event included in the plurality of coincidence events produces two coincident gamma ray interactions that are detected by the one or more PET detectors;
determine a line of response for the two coincident gamma ray interactions generated by each coincidence event;
measure, using the time of flight sensor, a time of flight for each coincident gamma ray interaction;
for each coincidence event, determine an annihilation position for a particular coincidence event along the line of response by measuring a difference between the time of flight for the two coincident gamma ray interactions produced during a particular coincidence event;
determine a distribution of annihilation positions over the volume of interest by counting a number of annihilation positions within each region in the plurality of regions; and
determine a current position of the tumor based on the distribution of annihilation positions.

12. The apparatus of claim 11, wherein the plurality of coincidence events are acquired during a measurement time frame having a duration of between 0.01 seconds and 0.2 seconds.

13. The apparatus of claim 11, wherein the current position of the tumor is continuously determined during a radiation therapy session to track a position of the tumor as the tumor moves within the patient.

14. The apparatus of claim 11, wherein the processor circuit is further configured to apply a count threshold to the number of annihilation positions within each region of the volume of interest to remove background noise that may reduce an accuracy of the current position of the tumor.

15. The apparatus of claim 11, wherein the processor circuit is further configured to:
transmit the current position of the tumor to a radiation source; and
control a position of the radiation source based on the current position of the tumor to deliver radiation directly to the tumor.

16. The apparatus of claim 11, wherein the line of response corresponds to a straight line drawing between two gamma rays detected by the one or more PET detectors.

17. The apparatus of claim 11, wherein the annihilation positions are three dimensional and include at least one coordinate indicating a location a vertical direction, horizontal direction, and depth direction relative to a geometric center of the patient.

18. The apparatus of claim 11, wherein the tumor is within an organ that moves during a respiratory activity of the patient.

19. The apparatus of claim 11, wherein the distribution of annihilation positions and the current position of the tumor are generated from the PET detection data in a timer period of less than a second and using fewer computational resources than are required to construct and image representation of the PET detection data.

20. The apparatus of claim 11, wherein the processor circuit is further configured to use the current position of the tumor to improve a quality of an image representation of the tumor generated using stationary PET imaging.
